(19) United States
(12) Patent Application Publication
(10) Pub. No.: US 2008/0147041 A1
(43) Pub. Date: Jun. 19, 2008

(54) Device for Providing a Change in a Drug Delivery Rate

(75) Inventor: Jorgen Smedegaard Kristensen, Vanlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/816,748

(22) PCT Filed: Feb. 27, 2006
(86) PCT No.: PCT/EP06/60296

§ 371 Date: Oct. 29, 2007

(30) Foreign Application Priority Data
Feb. 28, 2005 (DK) PA 2005 00309

(51) Int. Cl. A61M 5/14

(52) U.S. Cl. 604/506; 604/131

(57) ABSTRACT

The present invention relates to devices for providing an effective change in a drug delivery rate. In an exemplary embodiment the invention provides a drug delivery device using a method for changing a delivery rate for a drug from a first delivery rate to a second higher delivery rate, comprising the steps of: (a) Deliver the drug at the first delivery rate, (b) deliver the drug at a third delivery rate for a first period of time, the third delivery rate being higher than the second delivery rate, and (c) after the first period of time deliver the drug at the second delivery rate. By using a higher “bolus-like” third delivery rate for a first period of time it is possible relatively fast to fill up a depot corresponding to the new second delivery rate.

The present invention generally relates to methods and devices for providing an effective change in a drug delivery rate. In specific embodiment the invention relates to methods for obtaining rapid changes in basal plasma drug levels by administering the drug in accordance with a tailored infusion profile.

### BACKGROUND OF THE INVENTION

In the disclosure of the present invention reference is mostly made to the treatment of diabetes by infusion of insulin, however, this is only an exemplary use of the present invention.

Drug delivery devices for delivering a drug to a patient are well known and generally comprise a reservoir adapted to contain a liquid drug, a pump assembly for expelling a drug out of the reservoir. The drug may be administered either directly to the blood circulation (IV) or through the skin of the subject via a transcutaneous access device such as a soft cannula or a needle. For the treatment of diabetes relatively small portable infusion pumps are normally used for subcutaneous infusion of insulin.

Basically, infusion pumps can be divided into two classes. The first class comprises durable infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals. Such pumps are normally carried in a belt or pocket close to the body.

Addressing the above cost issue, several attempts have been made to provide a second class of drug infusion devices that are low in cost yet convenient to use. Some of these devices are intended to be partially or entirely disposable and may provide many of the advantages associated with an infusion pump without the attendant costs. For example, EP 1 177 802 discloses a skin-mountable drug infusion device which may have a two-part construction in which more expensive electronic components are housed in a reusable portion and the fluid delivery components are housed in a separable disposable portion (i.e. intended for single use only). U.S. Pat. No. 6,656,159 discloses a skin-mountable drug infusion device which is fully disposable.

The traditional durable pump may be worn in a belt at the waist of the user, this allowing the user to operate the pump by directly accessing the user interface on the pump, e.g. in order to change infusion rate or to program a bolus infusion. However, the pump may also be worn hidden under clothing this making operation more difficult. Correspondingly, it has been proposed to provide an infusion pump of the durable type with a wireless remote controller allowing the user to access some or all of the functionality of the pump, see for example U.S. Pat. No. 6,551,276, US 2005/0022274 and US 2003/0065308, which are hereby incorporated by reference, the latter disclosing an ambulatory medical device (MD) adapted to receive control messages from a communication device (CD). For a skin-mountable device, typically comprising an adhesive allowing the device to be attached directly to the skin of the user, a remote controller would appear even more desirable. Correspondingly, EP 1 177 802 and U.S. Pat. No. 6,740,059, which are hereby incorporated by reference, disclose semi-disposable and fully disposable infusion devices which are intended to be operated primarily or entirely by a wireless remote controller. As the delivery device thus does not have to be provided with a user interface such as a display and keyboard, the semi-disposable or disposable infusion can be provided more cost-effectively.

Irrespective of the type of drug delivery device used, several diseases are treated with drugs that are administered by infusion. An example of such infusion treatment is Continuous Subcutaneous Infusion of Insulin (CSII) also called pump treatment. Diseases such as diabetes mellitus, anti cancer treatment and severe infections may be treated using infusion treatment. In these techniques a constant infusion rate is often applied, however, this may not be optimal as the drug requirements may differ during the day and the therapy as such may benefit from variations in the drug levels in the patient.

CSII treatment of diabetes is considered the state of the art treatment of Insulin Dependent Diabetes Mellitus (IDDM or Type 1 diabetes). CSII treatment is also used in selected patients with non-insulin dependent diabetes (Type 2). CSII treatment involves two different dosing principles: A basal rate infusion that covers the body's basal need, and bolus infusions provided in connection with meals.

Various algorithms exist for calculating the bolus doses. These algorithms do not differ in principle from the algorithms used when calculating a dose to be given as an insulin injection, although in more recent infusion pumps also the bolus infusions can be tailored to have a desired time dependent profile. However, the injection therapy regarding basal insulin supplementations differs radically from the basal infusion with CSII as the whole insulin dose is determined at the injection time. Thus, the basal insulin level in the patient is given for the next 12-24 hours, whereas the insulin administered with CSII can be altered during this period causing differences in plasma insulin levels and the associated changes in effect on the body metabolism of various substrates, e.g. glucose, lipids.

The basal insulin requirements are individual as they are determined by the insulin sensitivity of the patient, the BMI of the patient and a number of exogenous and endogenous factors as well. For example, stress will cause an increase in adrenalin levels and this will lead to an increase in blood glucose, if not counteracted. Also increased body temperature will cause that basal insulin levels need to be increased. On the other hand physical activities will decrease the need for basal insulin. This means that in order to obtain a good control of blood glucose the plasma insulin levels should be changed accordingly.

The conventional way of adjusting the basal insulin supplementation in CSII is not optimal in situations where e.g. more rapid changes in plasma insulin levels are needed. The insulin infused will first enter into a subcutaneous depot and subsequently be released from this depot. This is a slow process as it takes several hours to obtain a new equilibrium in plasma simply just by raising or lowering the infusion rate. This is not optimal in situations where short (few hours) and precise changes in plasma insulin levels need to be obtained.

An example is counteracting the Dawn phenomenon seen in the early morning hours (caused by an increase in hormones that elevates blood glucose if not counteracted by insulin). If a conventional approach is applied either the new insulin equilibrium will be obtained several hours after the Dawn phenomenon has occurred (if the basal infusion rate is increased at the time of the occurrence of Dawn). If the infusion rate is increased before the Dawn phenomenon in an attempt to synchronise the patient is exposed to too high insulin levels in the period before the Dawn phenomenon occurs. In both cases suboptimal blood glucose is the outcome with both the risk of getting too high blood glucose levels and acutely more serious low blood glucose levels. This can be overcome with the present invention.

Having regard to the above, it is an object of the present invention to provide methods and devices whereby a new equilibrium in plasma drug levels during drug infusion therapy can be achieved more rapidly than hitherto—not just for the above-discussed examples but whenever it is desirable to change a drug plasma level during infusion treatment.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect a method is provided for changing a delivery rate for a drug from a first delivery rate to a second higher delivery rate, comprising the steps of: (a) Deliver the drug at the first delivery rate, (b) deliver the drug at a third delivery rate for a first period of time, the third delivery rate being higher than the second delivery rate, and (c) after the first period of time deliver the drug at the second delivery rate. By using a higher “bolus-like” third delivery rate for a first period of time it is possible relatively fast to fill up a depot corresponding to the new second delivery rate. In this way a much more rapid adaptation of the plasma drug level can be achieved compared to traditional methods used in CSII. The “bolus infusion” may be used to create a smooth transition to the new plasma level, however, to achieve an even faster adaptation to the new level, the bolus infusion may “overshoot”, thereby creating (to a desired degree) a transitional drug plasma level which is higher than the intended level corresponding to the new second delivery rate. The pump rate and infusion time corresponding to the bolus infusion may be set by the user or it may be pre-programmed or automatically set in accordance with the calculated bolus size.

When the term “deliver” is used in the context of the present invention, this term covers both “delivery from” and “delivery to” when not otherwise specified. Delivery may thus be from a device or system, or delivery may be to another device or system, or it may be to a live subject.

To compensate for an overshoot, after the first period of time the drug may be delivered at a fourth delivery rate for a second period of time, the fourth delivery rate being lower than the second delivery rate, the second delivery rate being higher than zero.

The term “rate” may indicate that drug delivery takes place at a constant rate, however, for a given period of time a defined delivery rate may vary to a certain extent, either due to the actual technology implemented (e.g. using pulsatile delivery) or due to other considerations. For example, for a given first or second delivery rate it may be relevant to infuse a given drug in a non-technology related pulsatile mode. Thus, in the context of the present invention, when it is defined that a certain rate is higher or lower than a reference rate, this would cover a situation in which the average rates for two periods differ, e.g. by more than 10, 20 or 30 percent.

In an exemplary embodiment of the invention, the third and fourth delivery rates are substantially constant, especially, the fourth delivery rate may be substantially zero.

The desired change in delivery rate may be implemented automatically, e.g. in accordance with a pre-programmed infusion profile, or the second higher delivery rate may be set manually by a user before changing the first delivery rate, e.g. when programming a temporary basal infusion profile.

Instead of using actual delivery rates as a means of communication between a user and a delivery device, communication may be based on setting plasma drug levels to be achieved in a subject, the corresponding delivery rates being calculated using a mathematical formula for distribution of drug within the subject.

In a further aspect a method is provided for changing a delivery rate for a drug from a first delivery rate to a second lower delivery rate, comprising the steps of: (a) Deliver the drug at a first delivery rate, (b) deliver the drug at a third delivery rate for a first period of time, the third delivery rate being lower than the second delivery rate, and (c) after the first period of time deliver the drug at the second delivery rate. In this way a much more rapid adaptation to a lower plasma drug level can be achieved compared to traditional methods used in CSII. The third delivery rate may be substantially constant, e.g. substantially zero. The method may comprise the further step of allowing a user to set the second lower delivery rate before the first delivery rate is changed.

In a yet further aspect a method is provided for changing a delivery rate for a drug from a first delivery rate to a second higher delivery rate within a time interval (e.g. for a temporary change of delivery rate), the first delivery rate being higher than zero, comprising the steps of: (a) Deliver the drug at the first delivery rate, (b) at the start of or before the time interval deliver the drug at a third delivery rate for a first period of time, the third delivery rate being higher than the second delivery rate, (c) after the first period of time deliver the drug at the second delivery rate, (d) at the end of or before the end of the time interval deliver the drug at a fourth delivery rate for a second period of time, the forth delivery rate being lower than the first delivery rate, and (e) after the second period of time deliver the drug at the first delivery rate. As appears, this method provides a combination of the above-described first two methods, i.e. for a time interval changing a delivery rate up and subsequently back. The third and fourth delivery rates may be substantially constant, especially, the fourth delivery rate may be substantially zero. To compensate for an overshoot, after the first period of time the drug may be delivered at a fifth delivery rate for a third period of time, the fifth delivery rate being lower than the second delivery rate. The fifth delivery rate may be substantially constant, especially, it may be substantially zero.

As appears from the above, when the time interval is known, it may be possible to decide whether the change in delivery rate should start within the time interval or outside the time interval. For example, starting or ending the change outside the time interval would allow the desired new level to be achieved within a greater part of the interval, e.g. delivery of the drug at the third delivery rate may begin before the beginning of the time interval, and delivery of the drug at the fourth delivery rate may begin after the end of the time interval. The method may comprise the further step of allowing a user to set the set the time interval and the second higher delivery rate before the first delivery rate is changed.

In a further aspect a method is provided for changing a delivery rate for a drug from a first delivery rate to a second lower delivery rate within a time interval, the second delivery rate being higher than zero, comprising the steps of: (a) Deliver the drug at a first delivery rate, (b) at the start of or before the time interval deliver the drug at a third delivery rate for a first period of time, the third delivery rate being lower than the second delivery rate, (c) after the first period of time deliver the drug at the second delivery rate, (d) at the end of or before the end of the time interval deliver the drug at a fourth delivery rate for a second period of time, the forth delivery rate being higher than the first delivery rate, and (e) after the second period of time deliver the drug at the first delivery rate. The third and fourth delivery rates may be substantially constant, especially, the third delivery rate may be substantially zero. After the second period of time deliver the drug at a fifth delivery rate for a third period of time, the fifth delivery rate being lower than the first delivery rate. Also the fifth delivery rate may be substantially constant, especially, it may be substantially zero. Delivery of the drug at the third delivery rate may begin before the start of the time interval. The method may comprise the further step of allowing a user to set the set the time interval and the second lower delivery rate before the first delivery rate is changed.

For all of the above-described methods, the first and second delivery rates may be substantially constant.

In a further aspect a method is provided for providing a percentage change in a delivery profile for a drug for a time interval, the delivery profile within the time interval comprising at least two delivery rates, the method comprising the steps of: (a) Deliver the drug according to an initial profile, (b) creating for the time interval a temporary profile having delivery rates corresponding to a set percentage of the delivery rates of the initial profile, and (c) deliver the drug in accordance with the temporary profile, wherein at least one raise in the delivery rate is in accordance with a method as defined above, and wherein at least one lowering of the delivery rate is in accordance with a method as defined above. The method may comprise the further step of allowing a user to set the set the time interval and the percentage before the first delivery rate is changed.

In a yet further aspect of the invention a drug delivery device is provided, comprising a reservoir adapted to contain a fluid drug, an expelling assembly adapted for cooperation with the reservoir to expel fluid drug from the reservoir to a subject via an outlet, input means configured to receive settings from a user, and processor means for controlling the expelling assembly, wherein the processor means are configured to control the expelling assembly in accordance with a method as defined and discussed above. The drug delivery device may be in the form of a small (e.g. pocket size) personal drug infusion pump adapted to be carried by the user.

In the context of the present application and as used in the specification and claims, the term processor covers any combination of electronic circuitry suitable for providing the specified functionality, e.g. processing data and controlling memory as well as all connected input and output devices. The processor will typically comprise one or more CPUs or microprocessors which may be supplemented by additional devices for support or control functions. For example, a transmitter or a receiver may be fully or partly integrated with the processor, or may be provided by individual units. Each of the components making up the processor circuitry may be special purpose or general purpose devices.

In an exemplary embodiment the drug delivery device comprises a first unit in which the reservoir, expelling assembly and processor means are arranged, and a second unit comprising the user input means, wherein the first and second units are adapted for wireless transmission of the received settings from the second to the first unit. The drug delivery device may further comprise a transcutaneous device unit, the transcutaneous device unit comprising a hollow transcutaneous device, a fluid port in fluid communication with the flexible cannula, and a mounting surface adapted for application to the skin of a subject. The first unit may further comprise coupling means allowing the first unit to be attached to the transcutaneous device unit, the expelling assembly being adapted for cooperation with the reservoir to expel fluid drug out of the reservoir and through the skin of the subject via the fluid port and the transcutaneous device.

The reservoir may be any suitable structure adapted to hold an amount of a fluid drug, e.g. a hard reservoir, a flexible reservoir, a distensible or elastic reservoir. The reservoir may e.g. be prefilled, user-fillable or in the form of a replaceable cartridge which again may be pre-filled or fillable. The expelling assembly may be of any desired type, e.g. a membrane pump, a piston-cylinder pump or a roller-tube pump. Advantageously, the processor means is adapted to receive flow instructions from a second unit, the second unit comprising a user interface allowing a user to enter flow instruction for subsequent transmission to the process unit, e.g. programming a basal infusion rate profile or a bolus. The first unit may be adapted to be implanted or the outlet may comprise or be adapted to connect to a transcutaneous access device, thereby allowing a fluid drug to be expelled out of the reservoir and through the skin of the subject via the transcutaneous access device.

The drug delivery devices (or medical systems) of the invention may further comprise a transcutaneous device unit comprising a transcutaneous device, e.g. access device or sensor device, a mounting surface adapted for application to the skin of a subject, e.g. an adhesive surface, wherein the transcutaneous device unit and the first unit are adapted to be secured to each other to form a combined device.

Irrespective of which form values are entered into a drug delivery system, either the entered or calculated values may be shown on a display of the above-described drug delivery devices. For example, a drug delivery device may comprise a display device adapted to graphically display (1) the actual infusion rates as a function of time, (2) only the intended first and second infusion rates as a function of time, or (3), as a function of time, calculated plasma drug levels to be achieved in a subject on the basis of actual drug delivery rates.

In a further aspect a method for providing a change in a basal plasma drug level in a subject is provided, comprising the steps of: (a) Providing a drug delivery device adapted to expel a fluid drug at a controlled rate from an outlet, (b) establishing a fluid communication between the outlet and the subcutaneous tissue of the subject, (c) delivering drug to the subcutaneous tissue in accordance with a delivery rate or profile, and (d) changing the delivery rate or profile for the drug in accordance with a method as defined and described above.

Further, a computer program product for carrying out the methods described above is provided, when said computer program product is run on a computer or a microprocessor.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other sub-stances in both solid (dispensed) or liquid form. In the description of the exemplary embodiments reference will be made to the use of insulin. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIGS. 1-3 shows in perspective views sequences of use for a first embodiment of a drug delivery device,

FIG. 4 shows perspective view of the interior of the reservoir unit of FIG. 1,

FIG. 5 shows a schematic representation of a process unit and a control unit,

FIGS. 6 and 7 show plasma insulin profiles and corresponding pump rate profiles responsible therefore for a typical prior art infusion pump,

FIGS. 8 and 9 show schematically pump rates related to plasma insulin levels,

FIGS. 10-13 show plasma insulin profiles and corresponding pump rate profiles responsible therefore for situations in which a user for a period of time wishes to raise the pump rate,

FIGS. 14-17 show plasma insulin profiles and corresponding pump rate profiles responsible therefore for situations in which a user for a period of time wishes to lower the pump rate,

FIGS. 18-20 show plasma insulin profiles and corresponding pump rate profiles responsible therefore for situations in which a user wishes to perform a dual wave bolus infusion, and

FIGS. 21A and 21B show in a non-assembled respectively assembled state a cannula unit and a reservoir unit for a further embodiment of a drug delivery device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

Before turning to the present invention per se, a system suitable to be used in combination therewith will be described, the system comprising a pump unit, a patch unit adapted to be used in combination with the pump unit, and a remote control unit for wireless communication with the pump unit. However, the present invention may be used in any system or unit in which the features of the present invention would be relevant, e.g. in a conventional durable infusion pump or system.

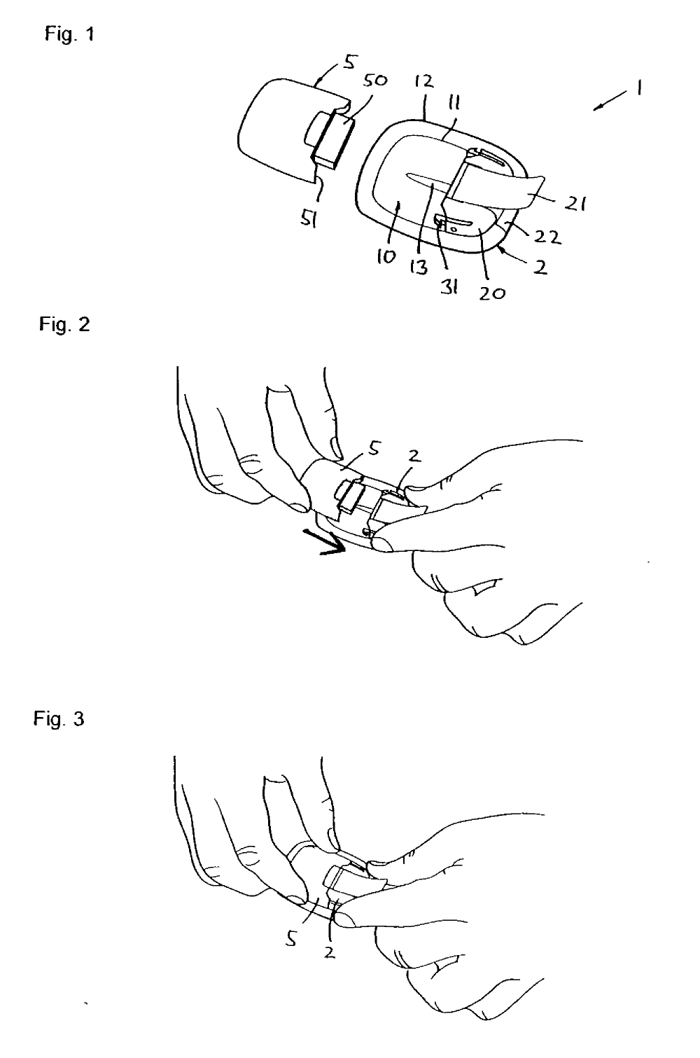

Firstly, with reference to FIGS. 1-3 an embodiment of a medical device for drug delivery will be described focusing primarily on the directly user-oriented features during application of the device to a skin surface. The patch unit 2 comprises a transcutaneous device in the form of a hollow infusion device, e.g. a needle or soft cannula, however, the needle or cannula may be replaced with any desirable transcutaneous device suitable for delivery of a fluid drug.

More specifically, FIG. 1 shows a perspective view of medical device in the form of a modular skin-mountable drug delivery device 1 comprising a patch unit 2 and a pump unit 5 (as the pump unit comprises a reservoir it may also be termed a reservoir unit). When supplied to the user each of the units are preferably enclosed in its own sealed package (not shown). The embodiment shown in FIG. 1 comprises a patch unit provided with an insertable transcutaneous device, e.g. needle, cannula or sensor. In case an actual embodiment requires the patch unit to be mounted on the skin and the transcutaneous device inserted before a pump or other unit can be attached, it follows that the method of use would be adopted correspondingly.

The patch unit comprises a flexible patch portion 10 with a lower adhesive mounting surface 12 adapted for application to the skin of a user, and a housing portion 20 in which a transcutaneous device (not shown) is arranged. The transcutaneous device comprises a pointed distal end adapted to penetrate the skin of a user, and is adapted to be arranged in fluid communication with the pump unit. In the shown embodiment the pointed end of the transcutaneous device is moveable between an initial position in which the pointed end is retracted relative to the mounting surface, and an extended position in which the pointed end projects relative to the mounting surface. The transcutaneous device may also be moveable between the extended position in which the distal end projects relative to the mounting surface, and a retracted position in which the distal end is retracted relative to the mounting surface.

The patch unit further comprises user-grippable actuation means in the form of a first strip-member 21 for moving the transcutaneous device between the initial and the second position when the actuation means is actuated, and a user-grippable second strip-member 22 for removing the patch from the skin surface. The second strip may also be used to move the distal end of the transcutaneous device between the extended and the retracted position. The housing further comprises user-actuatable male coupling means 31 in the form of a pair of resiliently arranged hook members adapted to cooperate with corresponding female coupling means 51 on the pump unit, this allowing the pump unit to be releasable secured to the patch unit in the situation of use. A flexible ridge formed support member 13 extends from the housing and is attached to the upper surface 11 of the patch. The adhesive surface is supplied to the user with a peelable protective sheet.

An alternative patch unit comprising an inserter mechanism for introducing a soft cannula is shown in co-owned PCT application EP2006/050410 which is hereby incorporated by reference. This alternative unit is adapted for mounting to a skin surface before the pump unit is attached, attachment of the pump unit releasing the inserter mechanism.

The pump unit 5 comprises a pre-filled reservoir containing a liquid drug formulation (e.g. insulin) and an expelling assembly for expelling the drug from the reservoir through the needle in a situation of use. The reservoir unit has a generally flat lower surface adapted to be mounted onto the upper surface of the patch portion, and comprises a protruding portion 50 adapted to be received in a corresponding cavity of the housing portion 20 as well as female coupling means 51 adapted to engage the corresponding hook members 31 on the needle unit. The protruding portion provides the interface between the two units and comprises a pump outlet and contact means (not shown) allowing the pump to detect that it has been assembled with the patch.

In a situation of use the user assembles the two units which are then mounted on a skin surface where after the transcutaneous device is inserted and the pump is ready to operate. Operation may start automatically as the transcutaneous device is inserted, or the pump may be started via the remote unit, see below. Before the pump unit is mounted to the patch unit, the user will normally have paired the pump unit with the remote unit, see below. In an alternative situation of use the user may first mount the patch unit to a skin surface and insert the transcutaneous device, after which the pump unit is mounted to the patch unit.

After the assembled device has been left in place for the recommended period of time for use of the patch unit (e.g. 48 hours)—or in case the reservoir runs empty or for other reasons—it is removed from the skin by gripping and pulling the retraction strip 22 which may also lead to retraction of the transcutaneous device. The pump unit may be removed from the patch unit before or after the patch unit is removed from the skin. Thereafter the pump unit can be used again with fresh patch units until it has been emptied or the patch has to be changed again.

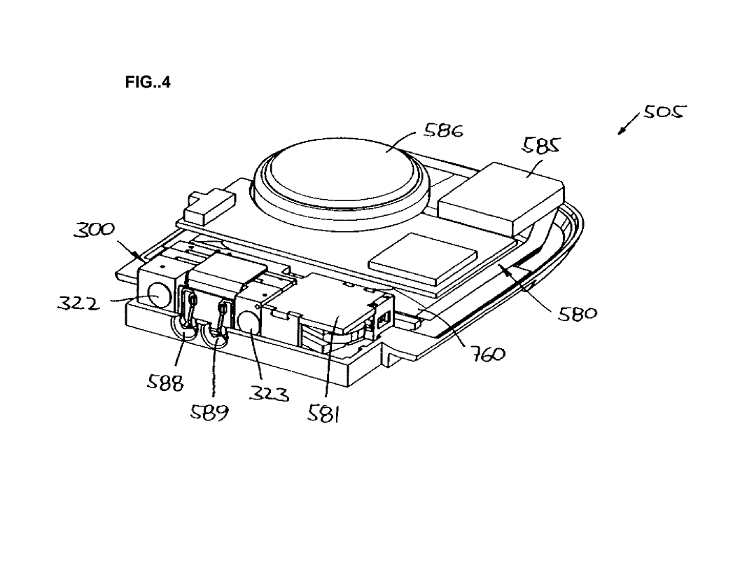

FIG. 4 shows the pump unit with an upper portion of the housing removed. The pump unit comprises a reservoir 760 and an expelling assembly comprising a pump assembly 300 as well as processor means 580 and a coil actuator 581 for control and actuation thereof. The pump assembly comprises an outlet 322 for connection to a transcutaneous access device and an opening 323 allowing a fluid connector arranged in the pump assembly to be actuated and thereby connect the pump assembly with the reservoir. The reservoir 560 is in the form of prefilled, flexible and collapsible pouch comprising a needle-penetratable septum adapted to be arranged in fluid communication with the pump assembly. The lower portion of the housing comprises a transparent area (not seen) allowing a user to inspect a portion of the reservoir. The shown pump assembly is a mechanically actuated membrane pump, however, the reservoir and expelling means may be of any suitable configuration.

The processor means comprises a PCB or flex-print to which are connected a microprocessor 583 for controlling, among other, the pump actuation, contacts (i.e. sensors) 588, 589 cooperating with corresponding contact actuators on the patch unit or the remote unit (see below), signal generating means 585 for generating an audible and/or tactile signal, a display (if provided), a memory, a transmitter and a receiver. An energy source 586 provides energy. The contacts may be protected by membranes which may be formed by flexible portions of the housing.

With reference to FIGS. 1-4 a modular local unit comprising a pump unit and a patch unit has been described, however, the local unit may also be provided as a unitary unit.

Although the present invention will be described with reference to the pump unit and the remote controller unit disclosed in FIGS. 1-5, it should be understood that the present disclosure is broadly applicable to any form of system providing drug delivery to a subject. For example, the present disclosure may be used with programmable ambulatory insulin infusion pumps of the sort currently commercially available from a number of manufacturers, including without limitation and by way of example, Medtronic MiniMed under the trademark PARADIGM, Insulet Corporation under the trademark OmniPod, Smiths Medical under the trademark Deltec COZMO, and others, these pumps either being provided with a remote control or being adaptable to be used with one.

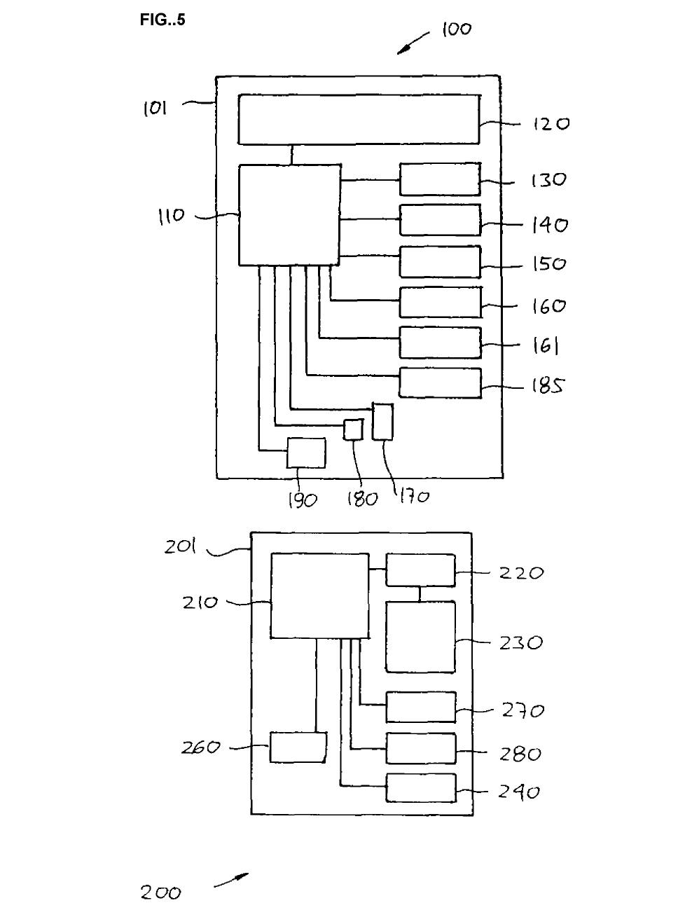

FIG. 5 shows a schematic representation of a process unit 200 (here corresponding to the pump unit 5 of FIG. 1) and a controller unit 100 (here in the form of a wireless “remote controller”or “external communication device” for the pump unit). It is considered that the general design of such units is well known to the skilled person, however, for a more detailed description of the circuitry necessary to provide the desired functionality of the present invention reference is made to incorporated US 2003/0065308.

More specifically, FIG. 5 depicts a simplified block diagram of various functional components or modules (i.e. single components or groups of components) included in the pump unit 200 and remote controller 100. The remote controller unit includes a housing 101, a remote processor 110 including a CPU, memory elements for storing control programs and operation data and a clock, an LCD display 120 for providing operation for information to the user, a keypad 130 for taking input from the user, an audio alarm 140 for providing information to the user, a vibrator 150 for providing information to the user, a main battery 160 for supplying power to the controller, a backup battery 161 to provide memory maintenance for the controller, a remote radio frequency (RF) telemetry transmitter 170 for sending signals to the pump unit, a remote radio frequency (RF) telemetry receiver 180 for receiving signals from the pump unit, and a second transmitter 190. The controller further comprises a port 185, e.g. an infrared (IR) or RF input/output system, or a USB port for communicating with a further device, e.g. a blood glucose meter (BGM), a continuous blood glucose meter (CGM), a PC or a PDA.

As also depicted in FIG. 5, the pump unit 200 includes a housing 201, local processor electronics 210 including a CPU and memory elements for storing control programs and operation data, battery 260 for providing power to the system, a process unit RF telemetry transmitter 270 for sending communication signals to the remote unit, a process unit radio frequency (RF) telemetry receiver 280 for receiving signals from the remote unit, a second process unit receiver 240 (which may be in the form of a coil of an acoustic transducer used in an audio alarm for providing feedback to the user), a reservoir 230 for storing a drug, and a pump assembly 220 for expelling drug from the reservoir through a transcutaneous device to the body of a patient. In alternative embodiments the pump unit may also comprise an LCD display for providing information to the user, a keypad for taking input from the user, and a vibrator or other tactile actuator for providing information to the user. RF transmission may be in accordance with a standard protocol such as Bluetooth®.

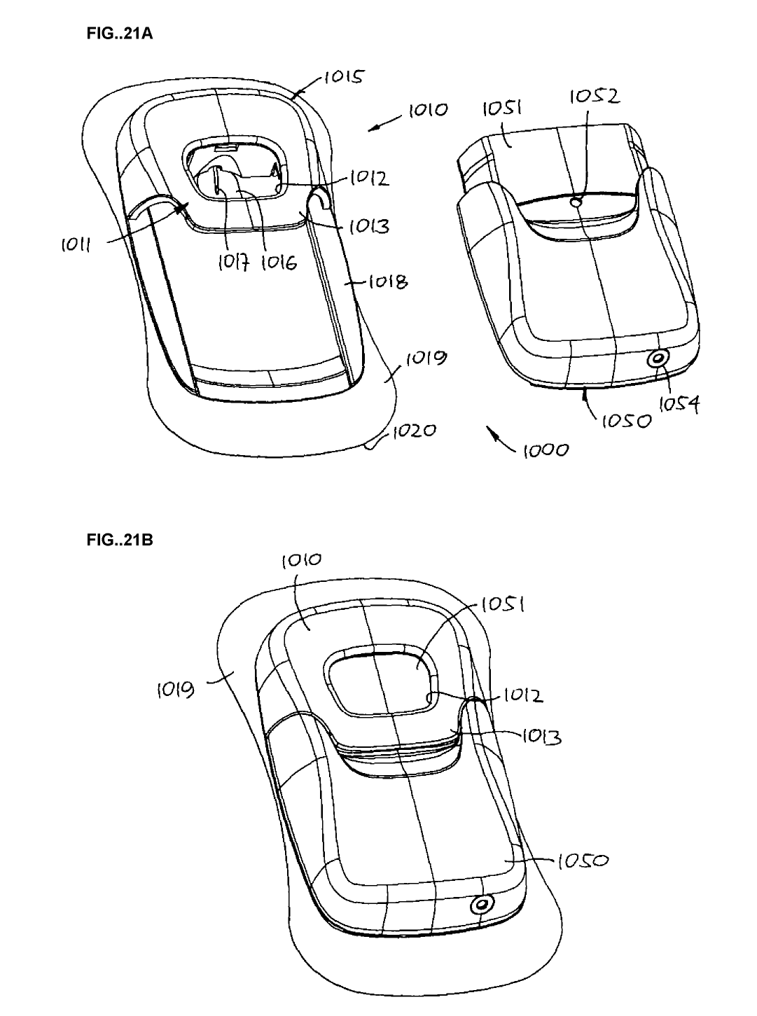

In FIG. 21A is shown an embodiment of a medical device 1000 of the type shown in FIG. 1, comprising a cannula unit 1010 and a thereto mountable pump (or reservoir) unit 1050, however, instead of a needle insertion mechanism as in the FIG. 1 embodiment, a cannula inserter mechanism as disclosed in PCT application EP2006/050410 is used. In the shown embodiment the cannula unit comprises a housing 1015 with a shaft into which a portion 1051 of the pump unit is inserted. The shaft has a lid portion 1011 with an opening 1012, the free end of the lid forming a flexible latch member 1013 with a lower protrusion (not shown) adapted to engage a corresponding depression 1052 in the pump unit, whereby a snap-action coupling is provided when the pump unit is inserted into the shaft of the cannula unit. Also a vent opening 1054 can be seen. The housing 1015 is provided with a pair of opposed legs 1018 and is mounted on top of a flexible sheet member 1019 with a lower adhesive surface 1020 serving as a mounting surface, the sheet member comprising an opening 1016 for the cannula 1017.

As appears, from the housing of the cannula unit a cannula extends at an inclined angle, the cannula being arranged in such a way that its insertion site through a skin surface can be inspected (in the figure the full cannula can be seen), e.g. just after insertion. In the shown embodiment the opening in the lid provides improved inspectability of the insertion site. When the pump unit is connected to the cannula unit it fully covers and protects the cannula and the insertion site from influences from the outside, e.g. water, dirt and mechanical forces (see FIG. 21B), however, as the pump unit is detachable connected to the cannula unit, it can be released (by lifting the latch member) and withdrawn fully or partly from the cannula unit, this allowing the insertion site to be inspected at any desired point of time. By this arrangement a drug delivery device is provided which has a transcutaneous device, e.g. a soft cannula as shown, which is very well protected during normal use, however, which by fully or partly detachment of the pump unit can be inspected as desired. Indeed, a given device may be formed in such a way that the insertion site can also be inspected, at least to a certain degree, during attachment of the pump, e.g. by corresponding openings or transparent areas, however, the attached pump provides a high degree of protection during use irrespective of the insertion site being fully or partly occluded for inspection during attachment of the pump.

In the shown embodiment an inclined cannula is used, however, in an alternative embodiment a needle mechanism of the type shown in FIG. 7 may be used if the point of insertion was moved closer to the coupling portion of the needle unit, this allowing also such a perpendicularly inserted to be inspected by detaching the pump unit.

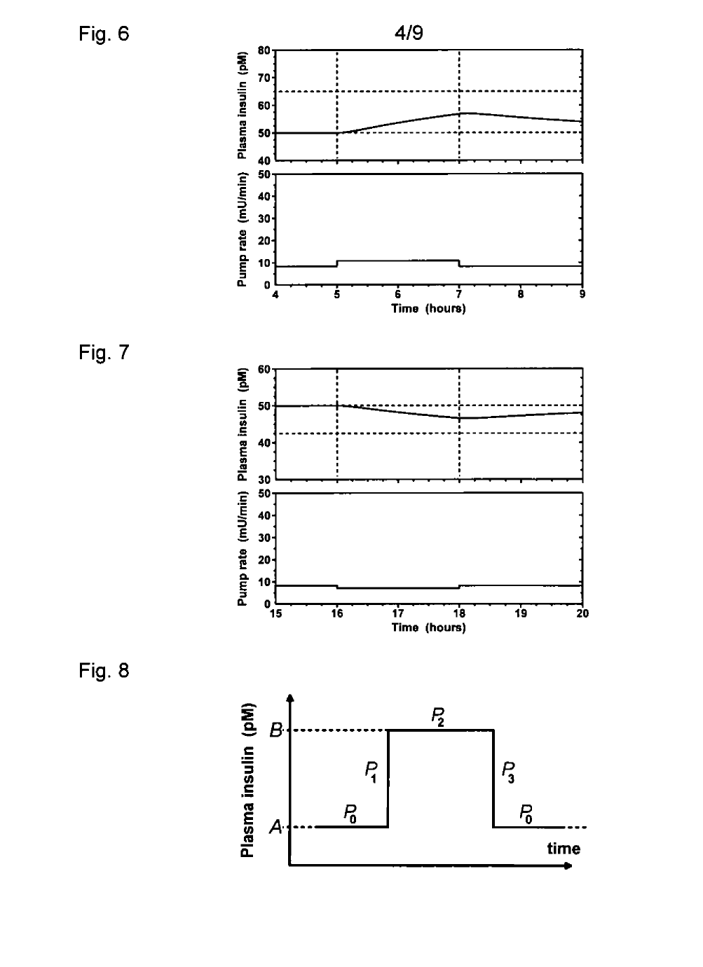

In the following aspects of the present invention will be described with reference to FIGS. 6-20. In each of FIGS. 6, 7 and 10-20 two diagrams are shown. A first diagram shows a plasma insulin profile achieved by a corresponding pump rate profile shown in a second diagram.

Before turning to the present invention, in FIGS. 6 and 7 plasma insulin profiles and corresponding pump rate profiles responsible therefore are shown for a typical prior art infusion pump. As appears, by simply raising (FIG. 6) or lowering (FIG. 7) the pump rate, the achieved plasma insulin profiles differ remarkably from what can be assumed to be the intended changes in the plasma insulin profile, i.e. as illustrated by the pump profiles. The achieved plasma insulin profiles are calculated using the below model and formulas.

In the following a simple two-compartment model for insulin delivery will be described, the model serving to illustrate the principles of the present invention. The model is characterized by the following components:
1) Injected subcutaneous depot corresponding to: P=pumping rate
2) Absorption to blood characterized by: T_{1/2}=absorption from depot
3) Plasma concentration characterized by: V=distribution volume
4) Elimination from blood characterized by: Cl=clearance from blood

For such a system the following model equations are applicable:

dD/dt=P(t)−k·D, k=ln(2)/T_{1/2}

dCₚ/dt=(k·D)/V−α·Cₚ, α=Cl/V

### Wherein:

D=dose or amount of insulin in depot (U)
P=pump rate (U/min)
k=time constant for absorption from depot (1/min)
Cₚ=plasma insulin concentration (U/L)
V=insulin distribution volume (L)
Cl=insulin clearance (L/min)

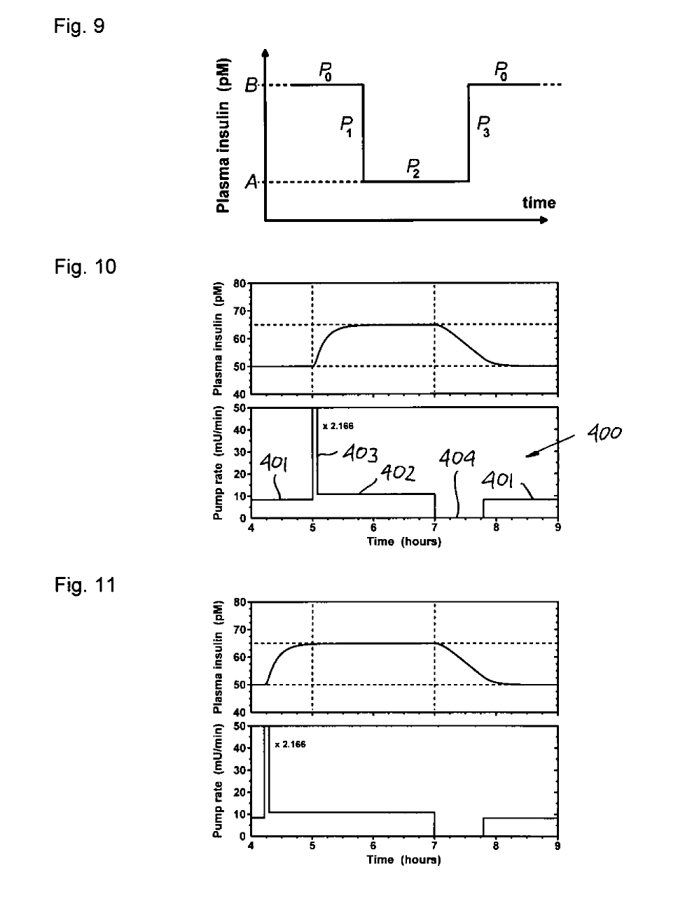

From the above equations it can be determined how to raise a plasma insulin level from level A, corresponding to infusion rate P₀, to level B, corresponding to infusion rate P₂, and to lower it back to level A, the infusion rates during the transition periods from A to B and from B to A being termed P₁ respectively P₃ (see FIG. 8).

P₀=Cl·A·n, n= 1/6000 (pmol/U)

P₁=P₀+P₁*=P₀+n·(B−A)·Cl/(k·Δt), Δt=n·(B−A)·Cl/(k·P₁*)

Wherein P₁* is a chosen pump rate and Δt is the corresponding time for building up the depot. After Δt the pump rate shifts to P₂.

P₂=Cl·B·n

P₃=0, Δt=−ln(A/B)/k

### Wherein Δt is the time for the depot size to change corresponding to a plasma level change from B to A.

From the above equations it can also be determined how to lower a plasma insulin level from level B, corresponding to infusion rate P₀, to level A, corresponding to infusion rate P₂, and to raise it back to level B, the infusion rates during the transition periods from B to A and from A to B being termed P₁ respectively P₃ (see FIG. 9).

P₀=Cl·B·n, n= 1/6000 (pmol/U)

P₃=P₂+P₃*=P₂+n·(B−A)·Cl/(k·Δt), Δt=n·(B−A)·Cl/(k·P₃*)

Wherein P₃* is a chosen pump rate and Δt is the corresponding time for building up the depot. After Δt the pump rate shifts to P₂.

P₂=Cl·A·n

P₁=0, Δt=−ln(A/B)/k

### Wherein Δt is the time for the depot size to change corresponding to a plasma level change from B to A.

In the following examples the following values have been used, however, it should be noted that the values are only illustrative as they will vary from person to person as well as over time for a given person:

T_{1/2}=126 min.

V=10 L

Cl=1 L/min

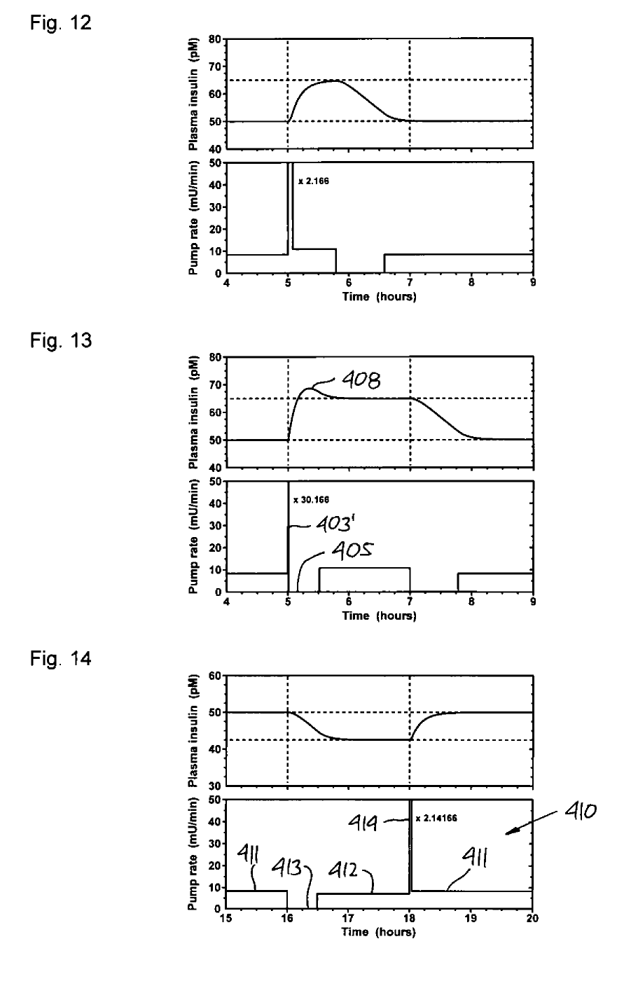

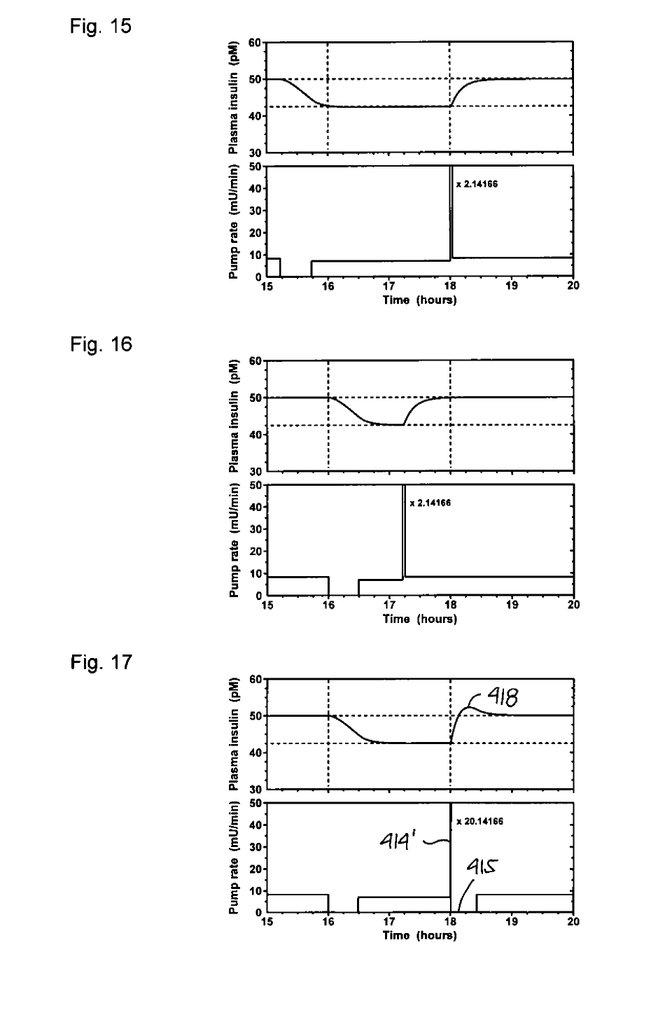

Turning to FIGS. 10-20 different methods for achieving a desired change in plasma insulin level is implemented. The achieved plasma insulin profiles are calculated using the above model and formulas. For very high pump rates a factor is shown which the shown pump rate of 50 mU/min has to be multiplied with. In the calculations on which the shown profiles are based, the plasma insulin has been used as a starting point, this resulting in non-integer infusion rates. However, traditional integer infusion rates may alternatively be set by the user. For example, in FIGS. 10-13 the pump rate is raised corresponding to a desired raise in plasma insulin of 30% from 50 pM to 65 pM, this corresponding to calculated infusion rates of approximately 8.33 and 10.83 mU/min. In FIGS. 14-17 the pump rate is lowered corresponding to a desired lowering in plasma insulin of 15% from 50 pM to 42.5 pM, this corresponding to calculated infusion rates of approximately 8.33 and 7.08 mU/min.

Thus, FIG. 10 illustrates a situation in which the user for a period of two hours wishes to raise the pump rate corresponding to a raise in plasma insulin of 30% from 50 pM to 65 pM, this corresponding to calculated infusion rates of 8.33 and 10.83 mU/min.

However, instead of merely raising the pump rate by 30% as illustrated in FIG. 6, the pump profile 400 is changed in accordance with an aspect of the present invention. More specifically, a method for providing a change in a delivery rate for a drug from a first delivery rate to a second higher delivery rate within a time interval is used (the first delivery rate being higher than zero), comprising the steps of: Deliver the drug at the first delivery rate (401), at the start of the time interval deliver the drug at a third delivery rate (403) for a first period of time, after the first period of time deliver the drug at the second delivery rate (402), at the end of the time interval deliver the drug at a fourth delivery rate (404) for a second period of time (here: zero), and after the second period of time again deliver the drug at the first delivery rate.

As appears in FIG. 10, a portion of the time interval is used to raise the plasma level to the desired level, just as the plasma level for a period of time after the time interval is higher than the initial plasma level. To compensate for this, and as shown in FIG. 11, the pump rate may be raised before the beginning of the time interval, this allowing the desired higher plasma level to be achieved within the entire time interval. Given that the time interval is known in advance, a processor controlled drug delivery system may calculate (using the above formulas) when infusion at the third infusion rate should begin. To avoid a raised plasma level after the end of the time interval, the pump rate may be lowered before the end of the time interval, this allowing the desired lower plasma level to be achieved after the time interval (see FIG. 12). As in the FIG. 11 situation, a processor controlled drug delivery system may calculate (using the above formulas) when infusion at the fourth infusion rate should begin.

In order to reach a plasma level corresponding to the second infusion rate faster, a higher third infusion rate 403′ may be used, this resulting in an “overshoot” 408 and a plasma level above the desired level. To compensate for such an overshoot, the pump rate may subsequently be lowered for a period of time to a fifth infusion rate 405 before being raised to the desired second pump rate, see FIG. 13. How fast the new level should be reached and how large an overshoot is acceptable can be selected as desired.

FIG. 14 illustrates a situation in which the user for a period of two hours wishes to lower the pump rate corresponding to a lowering in plasma insulin of 15% from 50 pM to 42.5 pM, this corresponding to calculated infusion rates of 8.33 and 7.08 mU/min. However, instead of merely lowering the pump rate by 15% as illustrated in FIG. 7, the pump profile 410 is changed in accordance with a further aspect of the present invention. In fact, it is the same principles used when the pump rate was raised for a time interval in the above example, the order of raising and lowering being exchanged. More specifically, a method for providing a change in a delivery rate for a drug from a first delivery rate to a second lower delivery rate within a time interval is used (the second delivery rate being higher than zero), comprising the steps of: Deliver the drug at a first delivery rate (411), at the start of the time interval deliver the drug at a third delivery rate (413) for a first period of time, the third delivery rate being lower than the second delivery rate (here: zero), after the first period of time deliver the drug at the second delivery rate (412), at the end of the time interval deliver the drug at a fourth delivery rate (414) for a second period of time, the forth delivery rate being higher than the first delivery rate, and after the second period of time deliver the drug at the first delivery rate.

As appears in FIG. 14, a portion of the time interval is used to lower the plasma level to the desired level, just as the plasma level for a period of time after the time interval is lower than the initial plasma level. To compensate for this, and as shown in FIG. 15, the pump rate may be lowered before the beginning of the time interval, this allowing the desired lower plasma level to be achieved within the entire time interval. Given that the time interval is known in advance, a processor controlled drug delivery system may calculate (using the above formulas) when infusion at the third infusion rate should begin. To avoid a lowered plasma level after the end of the time interval, the pump rate may be raised before the end of the time interval, this allowing the desired higher plasma level to be achieved after the time interval (see FIG. 16). As in the FIG. 15 situation, a processor controlled drug delivery system may calculate (using the above formulas) when infusion at the fourth infusion rate should begin.

In order to reach a plasma level corresponding to the initial first infusion rate faster after the end of the time interval, a higher fourth infusion rate 414′ may be used, this resulting in an “overshoot” 418 and a plasma level above the desired level. To compensate for such an overshoot, the pump rate may subsequently be lowered for a period of time to a fifth infusion rate 415 before being raised to the desired second pump rate, see FIG. 17. How fast the new level should be reached and how large an overshoot is acceptable can be selected as desired.

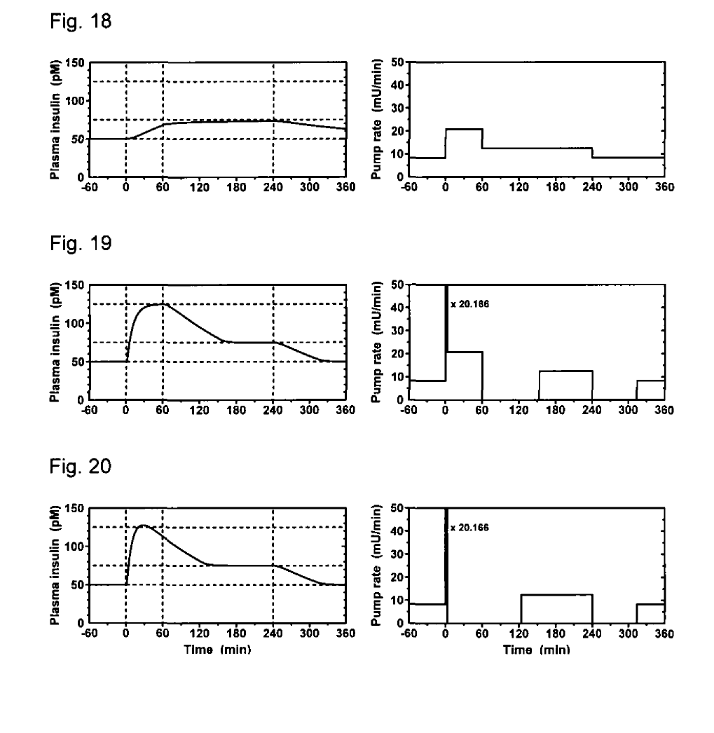

With reference to FIGS. 6 and 7 plasma insulin profiles and corresponding pump rate profiles responsible therefore were shown for a typical prior art infusion pump. FIG. 18 shows a corresponding example in which a “dual wave” bolus is infused over 2 hours. As appears, by simply raising and lowering the pump rate, the achieved plasma insulin profile differs remarkably from what can be assumed to be the intended changes in the plasma insulin profile, i.e. as illustrated by the pump profile.

In order to achieve a realized insulin plasma profile closer to the intended profile for a dual wave bolus infusion, principles of the present invention was used as illustrated in FIGS. 19 and 20. In fact, the examples correspond to a combination of the two above-described examples for raising respectively lowering an infusion rate. More specifically, in the FIG. 19 embodiment the principles implemented in the FIG. 10 embodiment is used, whereas in the FIG. 20 embodiment the principles implemented in the FIG. 13 embodiment is used, i.e. overshoot followed by subsequently compensating lowering of the infusion rate. As appears, the plasma levels achieved by the present invention as seen in FIGS. 19 and 20 are much closer to the intended dual wave profile.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A method for providing a change in a delivery rate for a drug from a first delivery rate to a second higher delivery rate, comprising the steps of:
deliver the drug at the first delivery rate (401),
deliver the drug at a third delivery rate (403) for a first period of time, the third delivery rate being higher than the second delivery rate, and
after the first period of time deliver the drug at the second delivery rate (402).

2. A method as in claim 1, comprising the further step of:
after the first period of time deliver the drug at a fourth delivery rate (405) for a second period of time, the fourth delivery rate being lower than the second delivery rate, the second delivery rate being higher than zero.

3. A method as in claim 2, wherein the third and fourth delivery rates are substantially constant.

4. A method as in claim 2, wherein the fourth delivery rate is substantially zero.

5. A method as in claim 1, comprising the further step of:
setting the second higher delivery rate before changing the first delivery rate.

6. A method as in claim 5, wherein the second delivery rate is set and calculated on the basis of setting a plasma drug level to be achieved in a subject.

7. A method for providing a change in a delivery rate for a drug from a first delivery rate to a second lower delivery rate higher than zero, comprising the steps of:
deliver the drug at a first delivery rate (411),
deliver the drug at a third delivery rate (413) for a first period of time, the third delivery rate being lower than the second delivery rate, and
after the first period of time deliver the drug at the second delivery rate (412).

8. A method as in claim 7, wherein the third delivery rate is substantially constant.

9. A method as in claim 7, wherein the third delivery rate is substantially zero.

10. A method as in claim 7, comprising the further step of:
setting the second lower delivery rate before changing the first delivery rate.

11. A method as in claim 10, wherein the second delivery rate is set and calculated on the basis of setting a plasma drug level to be achieved in a subject.

12. A method for providing a change in a delivery rate for a drug, comprising the steps of:
providing a raise in a delivery rate for a drug as defined in claim 1, and
providing a lowering of a delivery rate for a drug as defined in claim 7.

13. A method for providing a change in a delivery rate for a drug, comprising the steps of:
providing a lowering of a delivery rate for a drug as defined in claim 7, and
providing a raise in a delivery rate for a drug as defined in claim 1.

14. A method as in claim 12, wherein the delivery rate before and after the change in the delivery rate is substantially the same.

15. A method for providing a change in a delivery rate for a drug from a first delivery rate to a second higher delivery rate within a time interval, the first delivery rate being higher than zero, comprising the steps of:
deliver the drug at the first delivery rate (401),
at the start of or before the time interval deliver the drug at a third delivery rate (403) for a first period of time, the third delivery rate being higher than the second delivery rate,
after the first period of time deliver the drug at the second delivery rate (402),
at the end of or before the end of the time interval deliver the drug at a fourth (404) delivery rate for a second period of time, the forth delivery rate being lower than the first delivery rate, and
after the second period of time deliver the drug at the first delivery rate (401).

16. A method as in claim 15, wherein the third and fourth delivery rates are substantially constant.

17. A method as in claim 15, wherein the fourth delivery rate is substantially zero.

18. A method as in claim 15, comprising the further step of:
after the first period of time deliver the drug at a fifth delivery rate (405) for a third period of time, the fifth delivery rate being lower than the second delivery rate.

19. A method as in claim 18, wherein the fifth delivery rate is substantially constant.

20. A method as in claim 18, wherein the fifth delivery rate is substantially zero.

21. A method as claim 15, wherein delivery of the drug at the fourth delivery rate begins before the end of the time interval.

22. A method as in claim 15, comprising, before changing the first delivery rate, the further steps of:
setting the time interval,
setting the second higher delivery rate for the time interval.

23. A method as in claim 22, wherein the second delivery rate is set and calculated on the basis of setting a plasma drug level to be achieved in a subject.

24. A method for providing a change in a delivery rate for a drug from a first delivery rate to a second lower delivery rate within a time interval, the second delivery rate being higher than zero, comprising the steps of:
deliver the drug at a first delivery rate (411),
at the start of or before the time interval deliver the drug at a third delivery rate (413) for a first period of time, the third delivery rate being lower than the second delivery rate,
after the first period of time deliver the drug at the second delivery rate (412),
at the end of or before the end of the time interval deliver the drug at a fourth delivery rate (414) for a second period of time, the forth delivery rate being higher than the first delivery rate, and
after the second period of time deliver the drug at the first delivery rate (411).

25. A method as in claim 24, wherein the third and fourth delivery rates are substantially constant.

26. A method as in claim 24, wherein the third delivery rate is substantially zero.

27. A method as in claim 24, comprising the further step of:
after the second period of time deliver the drug at a fifth delivery rate (415) for a third period of time, the fifth delivery rate being lower than the first delivery rate.

28. A method as in claim 27, wherein the fifth delivery rate is substantially constant.

29. A method as in claim 27, wherein the fifth delivery rate is substantially zero.

30. A method as in claim 24, wherein delivery of the drug at the third delivery rate begins before the start of the time interval.

31. A method as in claim 24, comprising, before changing the first delivery rate, the further steps of:
setting a time interval,
setting the second lower delivery rate for the time interval.

32. A method as in claim 31, wherein the second delivery rate is set and calculated on the basis of setting a plasma drug level to be achieved in a subject.

33. A method as in claim 1, wherein the first and second delivery rates are substantially constant.

34. A method for providing a percentage change in a delivery profile for a drug for a time interval, the delivery profile within the time interval comprising at least two delivery rates, the method comprising the steps of:
deliver the drug according to an initial profile,
creating for the time interval a temporary profile having delivery rates corresponding to a set percentage of the delivery rates of the initial profile, and
deliver the drug in accordance with the temporary profile,
wherein at least one raise in the delivery rate is in accordance with a method as defined in claim 1, and
wherein at least one lowering of the delivery rate is in accordance with a method as defined in claim 7.

35. A method as in claim 34, comprising, before changing the initial profile, the further steps of:
setting the time interval, and
setting the percentage.

36. A method as in claim 1, comprising the further step of calculating at least one delivery rate on the basis of a plasma drug level to be achieved in a subject.

37. A drug delivery device (100, 200) comprising:
a reservoir (230) adapted to contain a fluid drug,
an expelling assembly (220) adapted for cooperation with the reservoir to expel fluid drug from the reservoir to a subject via an outlet,
input means (130) configured to receive settings from a user, and
processor means (110) for controlling the expelling assembly,
wherein the processor means are configured to control the expelling assembly in accordance with a method as defined in claim 1.

38. A drug delivery device as in claim 37, comprising:
a first unit in which the reservoir, expelling assembly and processor means are arranged, and
a second unit comprising the user input means, wherein
the first and second units are adapted for wireless transmission of the received settings from the second to the first unit.

39. A drug delivery device as in 38, further comprising a transcutaneous device unit, the transcutaneous device unit comprising:
a hollow transcutaneous device,
a fluid port in fluid communication with the flexible cannula,
a mounting surface adapted for application to the skin of a subject,
the first unit further comprising:
coupling means allowing the first unit to be attached to the transcutaneous device unit, the expelling assembly being adapted for cooperation with the reservoir to expel fluid drug out of the reservoir and through the skin of the subject via the fluid port and the transcutaneous device.

40. A drug delivery device as in claim 37, further comprising a display device adapted to graphically display the actual infusion rates as a function of time.

41. A drug delivery device as in claim 37, further comprising a display device adapted to graphically display only the intended first and second infusion rates as a function of time.

42. A drug delivery device as in claim 37, further comprising a display device adapted to graphically display, as a function of time, calculated plasma drug levels to be achieved in a subject on the basis of actual drug delivery rates.

43. A computer program product for carrying out the method according to claim 1 when said computer program product is run on a computer or a microprocessor.

44. A method for providing a change in a basal plasma drug level in a subject, comprising the steps of:
providing a drug delivery device adapted to expel a fluid drug at a controlled rate from an outlet,
establishing a fluid communication between the outlet and the subcutaneous tissue of the subject,
delivering drug to the subcutaneous tissue in accordance with a delivery rate or profile, and
change the delivery rate or profile for the drug in accordance with a method as defined in claim 1.
